(19) United States
(12) Patent Application Publication
(10) Pub. No.: US 2008/0139657 A1
(43) Pub. Date: Jun. 12, 2008

(54) METHOD FOR INCREASING SEROTONIN LEVELS IN A PERSON BY ADMINISTRATION OF A COMPOSITION INCORPORATING (-)HYDROXYCITRIC ACID, AND RELATED COMPOSITIONS THEREOF

(75) Inventors: Sunny E. Ohia, Omaha, NE (US); Harry G. Preuss, Fairfax, VA (US); Debasis Bagchi, Concord, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: INTERHEALTH NUTRACEUTICALS, INC., Benicia, CA (US)

(21) Appl. No.: 12/033,419

(22) Filed: Feb. 19, 2008

Related U.S. Application Data
(62) Division of application No. 10/473,557, filed on Apr. 6, 2004.
(60) Provisional application No. 60/280,593, filed on Mar. 30, 2001.
(60) Provisional application No. 60/343,473, filed on Dec. 20, 2001.

(51) Int. Cl. A61K 31/19; A61P 25/00

(52) U.S. Cl. 514/574

(57) ABSTRACT

A method for increasing serotonin levels in a person includes identifying a person having a deficient serotonin level and administering to the person a composition incorporating hydroxycitric acid, preferably in the form of an extract of Garcinia cambogia, in an amount sufficient to increase the person's serotonin levels. The method also can incorporate administering chromium, preferably in the form of oxygen-coordinated, niacin-bound chromium, and gymnemic acid, preferably in the form of an extract of Gymnema sylvestre, to synergistically work to further increase serotonin levels in the person.

This application is a divisional of U.S. patent application Ser. No. 10/473,557, filed Apr. 6, 2004, which is the national phase application under 35 U.S.C. 371 of International Patent No. PCT/US02/10368, filed Apr. 1, 2002, which claims priority from U.S. Provisional Application Nos. 60/280,593, filed Mar. 30, 2001, and 60/343,473, filed Dec. 20, 2001. The present invention relates generally to a method for increasing serotonin levels in a person. The present invention also relates to compositions that, when administered to a person, increase serotonin levels in the person.

### BACKGROUND OF THE INVENTION

Serotonin (or 5-hydroxytryptamine, 5HT) is a neurotransmitter believed to be involved a wide range of mental and physical functions in the body, including sleep, mood, and eating behavior. Serotonin deficiency has been implicated in a variety of conditions, including depression, low energy, anxiety, affective disorder, obsessive-compulsive behavior, overeating, insomnia, schizophrenia, migraine headaches and bulimia. It is well-established that serotonin and peptides such as neuropeptide Y are involved in the regulation of eating behavior. Increased brain levels of serotonin have been linked with appetite suppression in preclinical experiments in animals and in clinical studies with human patients. These conditions can be resolved or improved dramatically when serotonin levels of the affected person are increased.

Methods for increasing serotonin levels in persons suffering from serotonin deficiency have included use of serotonin selective re-uptake inhibitors, (e.g, fluoxetine), compounds promoting production of serotonin, (e.g., St. John's Wort), or compounds inhibiting the degradation of serotonin (e.g., monoamine oxidase inhibitor antidepressants). These products, while somewhat effective, do not provide ideal results in all cases, and they also may result in negative side effects in persons ingesting them.

It is apparent from the above that a need exists for improved methods and compositions for increasing serotonin levels in persons in a safe and convenient manner. The present invention fulfills this need and provides further related advantages.

### BRIEF DESCRIPTION OF THE DRAWINGS

FIG. 1 is a graphical representation of the effect of exposure to (−)-hydroxycitric acid (HCA) on the release of radiolabeled serotonin from isolated, superfused, rat brain cortex slices. Stimuli were applied as follows: potassium chloride (K+, 50 mM) standard response at fractions 5 and 6 (S₁) and HCA at fractions 13 and 14 (S₂). Fractions of the superfusate were collected at 6-minute intervals and analyzed for radioactivity as described herein.

FIG. 2 is a graphical representation of the effect of exposure to (−)-hydroxycitric acid (HCA) on radiolabeled serotonin release from isolated, superfused rat brain cortex: control (K+) and in the presence of HCA (10 μM-1mM). Vertical bars represent means ±S.E.M. The number of observations is in parenthesis.

FIG. 3 is a graphical representation of the increase in serum serotonin level, increase in loss of body weight, and increase in unconsumed food observed in persons treated using methods of the present invention: control (placebo), (−)-hydroxycitric acid (HCA), and HCA plus chromium and gymnemic acid (HCA+).

### SUMMARY OF THE INVENTION

The present invention resides in a method for increasing serotonin levels in a person, comprising identifying a person having or at risk for having a deficient serum serotonin levels, and administering to the person a composition incorporating an amount of (−)-hydroxycitric acid effective to increase the serotonin levels of the person.

Preferably, the amount of (−)-hydroxycitric acid administered is effective in increasing the serotonin levels in the person sufficient to suppress the appetite of or alleviate mood disorders in the person. Particular mood disorders preferably alleviated include depression, anxiety, affective disorders, premenstrual dysphoria, insomnia, sleep-wake disturbances, binge eating, bulemia and obsessive-compulsive disorders. The amount of (−)-hydroxycitric acid administered also may preferably be effective to increase serotonin levels in the person sufficient to increase energy expenditure by the person, or to promote decrease of the person's body weight. p The method preferably incorporates administration of an extract of Garcinia cambogia as a source of the (−)-hydroxycitric acid. The amount administered preferably is between about 2,700 and about 2,800 mg of (−)-hydroxycitric acid per day, in three approximately equal, increments, preferably between about 45 and 60 minutes prior to consumption of a meal. Preferred methods also incorporate administering an amount of chromium sufficient, in combination with the (−)-hydroxycitric acid, to increase serum serotonin level in the person, in a preferred daily dose of 400 mcg. This chromium preferably is in the form of a niacin-bound, and more preferably oxygen-coordinated, niacin-bound, chromium. Preferred methods also incorporate administering an amount gymnemic acid sufficient, in combination with the (−)-hydroxycitric acid, to increase serum serotonin level, in a preferred daily dose of 100 mg. The preferred source of gymnemic acid is an extract of Gymnema sylvestre.

The present invention also resides in a composition incorporating hydroxycitric acid, niacin-bound chromium, and gymnemic acid, in the preferred individual dosages discussed above (i.e., between about 900 and 930 mg (−)-hydroxycitric acid, about 133 mcg chromium, and about 33 mg gymnemic acid). Preferred compositions consist essentially of an extract of Garcinia cambogia, an extract of Gymnema sylvestre, and niacin-bound chromium, preferably in amounts to provide the amounts of hydroxycitric acid, niacin-bound chromium, and gymnemic acid discussed above (i.e., between about 1,500 and 1,550 mg of Garcinia cambogia, about 1.3 mg of niacin-bound chromium, and about 130 mg of extract of Gymnema sylvestre). The composition may be in the form of a pill, tablet, capsule, powder, lozenge, or gum, or liquid. The composition also may be in the form of a food or beverage, such as a food bar or shake.

Other features and advantages of the present invention should become apparent from the following detailed description of the invention, taken with the accompanying drawings, which illustrate the principles of the invention. DETAILED DESCRIPTION OF THE PREFERRED METHOD

The present invention resides in a method for increasing serotonin levels in persons and alleviating various conditions linked to serotonin deficiency in those persons. The method includes identifying a person who is or is at risk for having deficient serotonin levels and administering to the person a composition comprising a salt of (−)hydroxycitric acid (HCA) in an amount effective to alleviate the deficiency. The present invention also resides in a composition comprising HCA, chromium, and gymnemic acid, preferably as a composition consisting essentially of preferred sources of HCA, chromium, and gymnemic acid.

Ingestion of a salt of (−)hydroxycitric acid is known to suppress appetite, inhibit fat production and decreases body weight in animals and persons. HCA has been shown to reduce food intake in experimental animals, suggesting a role for this agent in the treatment of obesity. HCA is a competitive inhibitor of ATP-citrate lyase, an extra-mitochondrial enzyme involved in the initial steps of de novo lipogenesis. Consequently, HCA reduces the transformation of citrate into acetyl coenzyme A, a step necessary for the formation of fatty acids in the liver. In the presence of HCA, there is increased production of hepatic glucogen, which has been believed to activate glucoreceptors leading to a sensation of fullness and reduced appetite. This mechanism of appetite suppression, however, has never been proven. It also has been shown that HCA-induced increases in energy expenditure may account, at least in part, for the observed inhibitory effect of this anorectic agent on body weight gain in rats.

Despite the known properties of HCA and its action at the metabolic level, no study has investigated its possible effect on neurotransmitters associated with the control of appetite and eating behavior. Recently, it has been found that consumption of HCA by persons increases their serum serotonin levels, reduces their appetites, and decreases their food intake. This increase in serotonin levels also may prove beneficial in addressing the other conditions known to be affected by low serotonin levels, including depression, low energy, anxiety disorder, obsessive-compulsive behavior, insomnia, schizophrenia, migraine headaches, and bulimia.

A preferred known composition incorporating HCA for use in the methods of the present invention is an extract of the Garcinia cambogia fruit containing approximately 60% calcium/potassium salt of (−)hydroxycitric acid, marketed under the name Super CitriMax™ by InterHealth Nutraceuticals of Benicia, Calif. This extract is highly soluble in water, and it is readily absorbed and retained by persons. Studies have shown that blood levels of the extract increase for at least 2 hours and remained in the blood for more than 4 to 9 hours after ingestion. Studies also show that eating a full meal shortly after consuming the extract reduced its absorption by about 60%. Thus, it is recommended that compositions containing the extract be taken at least 30 to 60 minutes before meals to provide maximum efficacy.

Preferred aspects of the method of the present invention incorporate administering compositions comprising chromium, gymnemic acid, or both of these. It has been surprisingly determined that consumption by persons of HCA in combination with these compounds provides for even greater increases in serotonin levels than consumption of HCA alone.

Chromium incorporated into the compositions used in the method of the present invention preferably is in an oxygen-coordinated, niacin-bound form. This form of chromium is known to be more bioavailable and biologically active that other known forms. A preferred source of this chromium is described in U.S. Pat. Nos. 4,934,855, 4,954,492, and 5,194,615 and is supplied by InterHealth Nutraceuticals, marketed under the name ChromeMate® ChromeMate® has been shown to promote weight loss and loss of body fat in persons ingesting it, with no adverse effects observed from this ingestion. No prior studies on chromium, however, have determined or suggested increases in serotonin levels from ingestion of chromium, either alone or in combination with other compounds.

Gymnemic acid has been shown to increase the production of insulin by stimulating the production of new insulin-promoting “beta-cells” cells in the pancreas. Gymnemic acid also facilitates insulin release from the beta-cells into the blood stream by increasing beta-cell membrane permeability, and inhibits the absorption of sugar molecules in the intestines during digestion, thus reducing increases in blood sugar levels. A preferred source of gymnemic acid in compositions used with the method of the present invention is Gymnema sylvestre extract supplied by InterHealth Nutraceuticals of Benicia, Calif. Gymnema sylvestre is a traditional Ayurvedic herb that is known to play a role in weight control by helping to promote normal blood sugar levels and reduce sugar cravings. Gymnema sylvestre also has also been shown to lower cholesterol in animal models. Despite its known properties, gymnemic acid or the Gymnema sylvestre previously have not been determined to affect serotonin levels in persons ingesting them.

Particularly referred methods of the present invention include administration of a composition incorporating between approximately 2,700 and 2,800 mg of HCA daily. Preferred administration of the composition is orally, in three daily doses roughly 30 to 60 minutes before meals. Additional preferred methods include administration of a composition further incorporating approximately 100 mg of gymnemic acid, or approximately 400 mcg of chromium, or both of these. As discussed above, the preferred source of gymnemic acid is Gymnema sylvestre extract. Approximately 400 mg of Gymnema sylvestre extract serves as a source of 100 mg of gymnemic acid. The preferred source of chromium is ChromeMate™, the oxygen-coordinated, niacin-bound chromium previously discussed. Approximately 4 mg of ChromeMate™ serves as a source of 400 mcg of chromium.

Methods of the present invention also include administration of compositions incorporating inert ingredients or diluents, such as sugar or other inert ingredients commonly used in food products. The composition administered may be in various forms commonly used for dietary supplements, including pill, tablet, capsule, powder, lozenge, gum, or liquid. The step of administering can include administering the compositions as part of functional foods and beverages, including bars, shakes, drinks, and other processed or prepared foods or beverages.

### EXAMPLES

Both preclinical and clinical studies were conducted to determine the efficacy of the methods and compositions of the present invention. The studies and their results are discussed in turn below.

1. Preclinical Study of Serotonin Increase from HCA

A study was conducted to evaluate the effect of HCA on brain serotonin levels. The aim of the study was to examine the effect of HCA on the release of radiolabeled serotonin from rat brain cortex slices in vitro.

a. Methods

Methods previously known for studies of radiolabeled serotonin release were employed. Isolated rat brain cortex slices were incubated in oxygenated Krebs buffer solution containing 800 nM radiolabeled serotonin, the monoamine oxidase inhibitor pargyline (10 μM), and the cyclooxygenase inhibitor flurbiprofen (3 μM) at 37° C. A natural extract of 60% (−)-hydroxycitric acid (HCA) from Garcinia cambogia (commercially known as Super CitriMax™, from InterHealth Nutraceuticals) was used. Radiolabeled serotonin was purchased from NEN Life Sciences, Boston, Mass. The Krebs solution used had the following composition (millimolar): potassium chloride, 4.8; sodium chloride 118; calcium chloride, 1.3; potassium dihydrogen phosphate, 1.2; sodium bicarbonate, 25; magnesium sulfate, 2.0; and dextrose, 10 (pH 7.4).

After incubation, tissues were rinsed, mounted between nylon mesh-cloth and placed in thermostatically-controlled superfusion chambers. Tissues were superfused at a rate of 0.5 ml/min with oxygenated Krebs solution containing clomipramine (10 μM), a serotonin reuptake inhibitor. Fractions of the superfusate were collected at 6-minute intervals, and 3-ml aliquots of each fraction was combined with 12 ml of aqueous scintillation cocktail marketed under the name Ecolume, by ICN Radiochemicals of California) and analyzed for radioactivity by liquid scintillation spectrometry.

After an initial 2 hours of superfusion to establish a stable baseline of spontaneous tritium efflux, release of radiolabeled serotonin was elicited by consecutive potassium-depolarizing (K+, 50 mM) stimuli applied at 144 minutes (S₁) and at 198 minutes (S₂) after the onset of superfusion. In some experiments, tissues were exposed to different concentrations of HCA for 12 minutes before the second K+ stimuli at S₂. When HCA was tested on its own, the K+(50 mM; S₁) peak was used as the standard (or control) response. In this case, effects induced by HCA on radiolabeled serotonin release were then compared with the standard K+ response. Both K+ and HCA-induced radiolabeled serotonin release were estimated by subtraction of the extrapolated basal tritium efflux from total tritium release in the 20-minute period after the onset of stimulation. Basal (unstimulated) tritium efflux was assumed to decline linearly between pre-stimulation and post-stimulation fractions. Stimulation-evoked radiolabeled serotonin release during S₁ and S₂ was determined graphically, and the ratio of the two peaks (S₁/S₂) was calculated and compared with untreated control preparations.

Results obtained were expressed as absolute S₁/S₂ ratios. Data from different experiments (control and test) were pooled and then subjected to statistical analysis. Except where indicated otherwise, values given are arithmetic means±SEM. Significance of difference between control and test values was evaluated using analysis of variance (ANOVA) followed by Dunnett's test. Differences with P values <0.05 were accepted as statistically significant.

b. Results

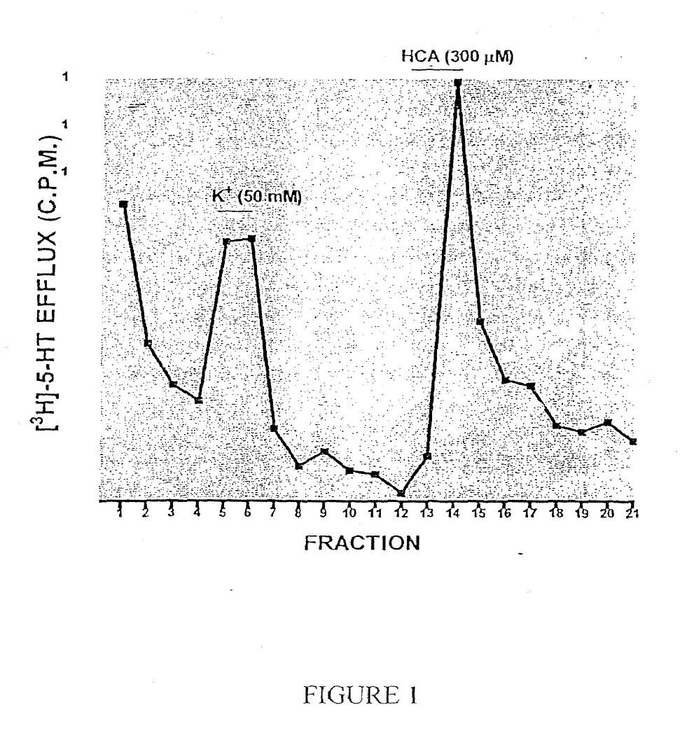

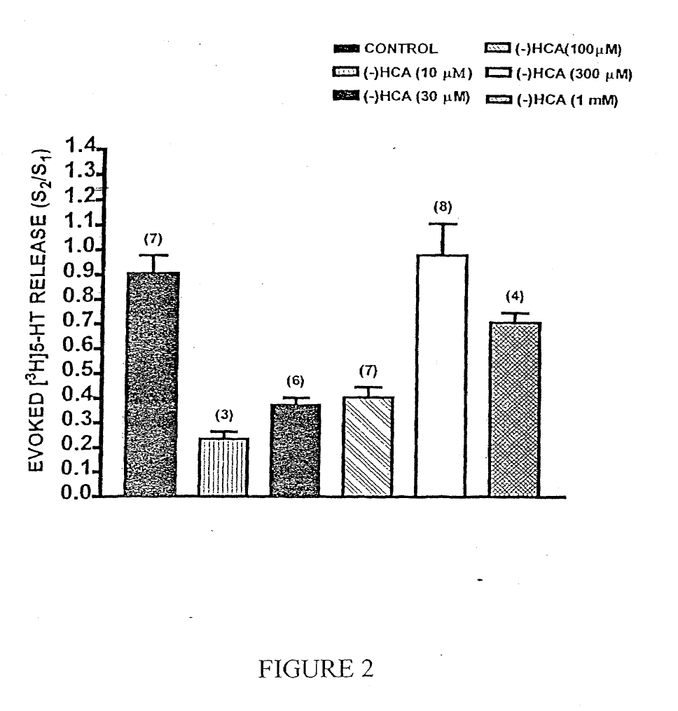

Results of the experiment are illustrated in FIGS. 1 and 2. Application of an iso-osmotic concentration of K+ (50 mM) elicited a peak of overflow of radiolabeled serotonin release, an effect that can be repeated more than twice in the same slice of brain cortex. The ratio of the size of the first (Si) and second (S₂) peaks of stimulation was 0.91±0.07 (n=7) indicating that there was no significant depletion of neurotransmitter occurring between stimuli. In preliminary experiments, the effect of different concentrations of HCA (10 μM-1 mM) applied 12 minutes before the second K+ stimuli (S₂) were examined. At these concentrations, HCA had no significant effect on the second K+ response even though it changed the baseline of spontaneous radiolabeled serotonin efflux. Consequently, the direct effect of HCA on basal radiolabeled serotonin release from brain cortex slices was investigated. For these experiments, the K+ stimulus was applied at S₁, and then the effect of HCA on basal tritium efflux was tested at fraction number 12. As represented by the illustration in FIG. 1, HCA (300 μM) elicited an increase in the release of radiolabeled serotonin over baseline values. Next, the effect of different concentrations of HCA (10 μM-1 mM) on basal release of radiolabeled serotonin from cortical slices was examined. HCA caused a concentration-related increase in basal efflux of radiolabeled serotonin, reaching a maximum at 300 μM (FIG. 2). The overflow of radiolabeled serotonin induced by the maximal concentration of HCA (300 μM) was equivalent to the release induced by the standard concentration of K+ (50 mM).

c. Discussion

The results of this study determined that HCA alters the release and/or availability of serotonin in the brain slices, specifically by increasing the release of serotonin from neuronal stores in the brain cortex in a concentration-dependent fashion. Exposure of the rat brain cortex slices to different concentrations of HCA had no significant effect on K+-depolarization evoked release of radiolabeled serotonin. However, on its own, HCA increased the basal release of tritium-labeled 5-HT in a concentration-dependent fashion. The maximal effect caused by HCA on radiolabeled serotonin release was equivalent to responses elicited by the K+ depolarizing stimuli. The exact mechanism whereby HCA induces an increase in basal release of radiolabeled serotonin from rat brain cortex slices is unknown. HCA may act via a “reserpine-like” or “tyramine-like” action to increase the efflux of radiolabeled pools of 5-HT in the brain cortex. A reserpine-like action may involve HCA induced interference with the storage of radiolabeled serotonin in vesicles whereas, a tyramine-like effect could involve vesicular release of radiolabeled serotonin in a non-exocytoxic manner. It is also feasible that HCA may act to prevent the reuptake of released radiolabeled serotonin, resulting in an increased efflux of this amine into the superfusate.

### 2. Clinical Study

The effects of administering compositions within the scope of the present invention were tested. A double-blind, placebo-controlled human clinical trial was conducted using a composition incorporating: the HCA extract described above, or the HCA extract in combination with an oxygen-coordinated niacin-bound chromium (ChromeMate®), supplied by InterHealth), and a standardized Gymnema sylvestre extract (also supplied by InterHealth).

a. Methods

Approximately 80 moderately obese human subjects were enrolled in the study. All subjects were placed on a daily diet of 2,000 kcal, weighing 2,250 grams. All food was prepared and delivered to the subjects, and all food intake was strictly supervised by trained dieticians. All subjects also underwent a 30 minute walking exercise program, five times a week, which was supervised by a trained exercise specialist.

The subjects were randomly divided into three groups. The first group was given a placebo. The second group was given a daily dose of 4,667 mg of garcinia cambogia extract (providing 2,800 mg HCA per day). The third group was given a daily dose of 4,667 mg of a combination of garcinia cambogia (2,800 mg HCA), 4 mg of niacin-bound chromium (providing 400 mcg of elemental chromium), and 400 mg of Gymnema sylvestre extract (providing 100 mg gymnemic acid). The subjects received their respective compositions in three equally-divided doses 30 to 60 minutes before breakfast, lunch and dinner for eight weeks. These dosage levels of HCA were determined by extrapolation of successful earlier animal trials, as well as review of optimal micromolar concentrations of HCA in ex vivo brain tissue resulting in maximum serotonin release.

The persons were assessed for changes in serum serotonin levels, body weight, and food intake. As discussed above, increases in serum serotonin levels relate to reduced appetite. Food intake was measured by monitoring the amount of food left unconsumed after each meal by the subjects. The amount of food left unconsumed while taking either the placebo or either of the HCA compositions was compared to the amount of food left unconsumed before the subjects began taking the compositions (i.e., the baseline). Changes in each of these factors were measured in the persons and averaged to produce the figures in Table 1.

b. Results

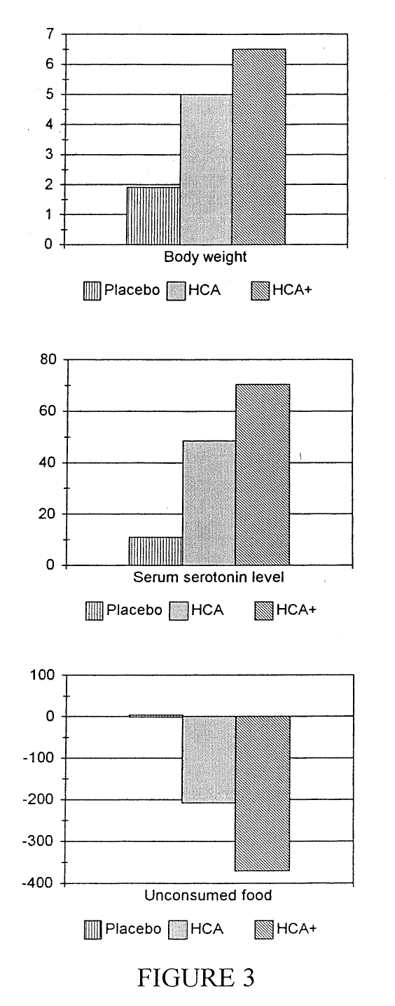

Results of the testing are shown in Table 1 below and FIG. 3.

TABLE 1
Results of Administration of Compositions

| | | | HCA + chromium + |
|---|---|---|---|
| Tested Factor | Placebo | HCA | gymnemic acid |
| Body weight | | | |
| Pounds lost | 3.5 | 10.0 | 12.8 |
| % decrease | 19. | 5.0 | 6.5 |
| Serum serotonin level | | | |
| Mg/dl increase | 20.1 | 119.1 | 149.3 |
| % increase | 10.9 | 48.5 | 70.4 |
| Food left unconsumed | | | |
| Grams | 71.9 | 249 | 328 |
| % increase from baseline | (3.6) | 206 | 370 |

In addition to the results noted above, no adverse effects were observed in the patients ingesting the compositions of the study.

c. Discussion

The data from this Example indicate that administration of the specified levels of HCA extract results in increases in serotonin levels and in related effects on body weight and food consumption. Specifically, serum serotonin levels, as shown in FIG. 3, increased almost 50 percent in subjects consuming HCA alone, compared to an increase of 10 percent in those consuming a placebo. Serotonin levels rose more dramatically, approximately 70 percent, in subjects consuming HCA in combination with chromium and gymnemic acid. Additionally, this increase in serotonin level led to increased losses of body weight and decreased food intake. Body weight losses increased two- to three-fold in subjects consuming the active compositions, compared to those consuming the placebo.

The results of the two studies indicate that consumption of HCA by persons can lead to increased serotonin levels in those persons, resulting in alleviation of conditions relating to low serotonin levels. Consumption of HCA can also be incorporated into a method of increasing serotonin levels in persons to alleviate any other conditions caused by serotonin deficiency. For example, as discussed above, current therapies for depression, insomnia, and migraine headaches involve increasing the serotonin levels in the affected persons. Consumption of a sufficient amount of HCA by a person suffering from depression, insomnia, or migraine headaches could raise serotonin levels and therefore eliminate these symptoms. Additionally, combining consumption of HCA with oxygen-coordinated niacin-bound chromium (incorporating elemental chromium), and Gymnema sylvestre extract (incorporating gymnemic acid), provides for increased efficacy of the method, increasing serum serotonin levels greater than consumption of HCA alone, further improving alleviation of the negative conditions discussed above. Administration of the three compounds works synergistically to substantially increase serotonin levels in persons consuming the compounds.

Although the invention has been disclosed in detail with reference only to the preferred embodiments, those skilled in the art will appreciate that additional methods and compositions can be made without departing from the scope of the invention.

We claim:

1. A method for increasing serotonin levels in a person, comprising:
identifying a person having or at risk for having deficient serotonin levels; and
administering to the person a composition comprising an amount of (−)-hydroxycitric acid effective to increase serotonin levels in the person.

2. A method as defined in claim 1, wherein the step of administering comprises administering an amount of (−)-hydroxycitric acid effective to increase serotonin levels in the person sufficient to suppress the appetite of the person.

3. A method as defined in claim 1, wherein the step of administering comprises administering an amount of (−)-hydroxycitric acid effective to increase serotonin levels sufficient to alleviate mood disorders in the person.

4. A method as defined in claim 3, wherein the mood disorders include depression, anxiety, affective disorders, premenstrual dysphoria, insomnia, sleep-wake disturbances, binge eating, bulimia, and obsessive-compulsive disorders.

5. A method as defined in claim 1, wherein the step of administering comprises administering an amount of (−)-hydroxycitric acid effective to increase serotonin levels in the person sufficient to increase energy expenditure by the person.

6. A method as defined in claim 1, wherein the step of administering comprises administering an amount of (−)-hydroxycitric acid effective to increase serotonin levels in the person sufficient to promote decrease of body weight of the person.

7. A method as defined in claim 1, wherein the step of administering comprises administering a composition comprising an extract of Garcinia cambogia.

8. A method as defined in claim 1, wherein the step of administering comprises administering between about 2,700 and about 2,800 mg of (−)-hydroxycitric acid per day.

9. A method as defined in claim 1, wherein the step of administering comprises administering the composition in three approximately equal increments per day.

10. A method as defined in claim 1, wherein the step of administering comprises administering the composition between about 45 and about 60 minutes prior to consumption of a meal by the person.

11. A method as defined in claim 1, wherein the step of administering comprises administering a composition comprising an amount of chromium sufficient, in combination with the amount of (−)-hydroxycitric acid, to increase serotonin levels in the person.

12. A method as defined in claim 11, wherein the step of administering comprises administering a composition comprising niacin-bound chromium.

13. A method as defined in claim 12, wherein the step of administering comprises administering a composition comprising oxygen-coordinated, niacin-bound chromium.

14. A method as defined in claim 11, wherein the step of administering comprises administering about 400 mcg of chromium per day.

15. The method of claim 1, wherein the step of administering comprises administering a composition comprising an amount of gymnemic acid sufficient, in combination with the amount of (−)-hydroxycitric acid, to increase serotonin levels in the person.

16. A method as defined in claim 15, wherein the step of administering comprises administering a composition comprising an extract of Gymnema sylvestre.

17. A method as defined in claim 15, wherein the step of administering comprises administering about 100 mg of gymnemic acid per day.
